(19) United States
(12) Patent Application Publication
(10) Pub. No.: US 2010/0087795 A1
(43) Pub. Date: Apr. 8, 2010

(54) SELF-LEARNING DRUG DELIVERY SYSTEM

(75) Inventors: Hendrika Cecilia Krijnsen, Eindhoven (NL); Ventzeslav Petrov Iordanov, Eindhoven (NL); Anna-Maria Janner, Eindhoven (NL); Michel Paul Barbara Van Bruggen, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS N.V., EINDHOVEN (NL)

(21) Appl. No.: 12/519,803

(22) PCT Filed: Dec. 19, 2007
(86) PCT No.: PCT/IB2007/055219

§ 371 Date: Jun. 18, 2009

(30) Foreign Application Priority Data
Dec. 22, 2006 (EP) 06127037.7

(51) Int. Cl. A61M 37/00; A61K 31/485; A61K 31/445; A61K 31/164; A61P 25/04

(52) U.S. Cl. 604/500; 514/282; 514/315; 514/626; 604/65

(57) ABSTRACT

Self-learning process for automatically adjusting a dosage of a substance administered to a person by a drug delivery device, a drug delivery device, wherein a dosage of a substance is adjusted by such a self-learning process and a drug delivery system comprising such a drug delivery device. Pain is experienced differently at certain times of day due to the origin causing the symptoms. Therefore, the medication providing pain relief should also be adjusted accordingly. The invention is to anticipate the pain experience and to deliver pain relief medication in such a way that Quality of Life is improved by appropriate timing of the right amount of medication and by appropriate timing of turning off the medication to reduce side effects.

### FIELD OF THE INVENTION

The present invention relates to a self-learning process for automatically adjusting a dosage of a substance administered to a person by a drug delivery device, to a drug delivery device in which a dosage of a substance is adjusted by such a self-learning process, and to a drug delivery system comprising such a drug delivery device.

### BACKGROUND OF THE INVENTION

Differences in pain experience do exist and the physiological basis for the temporal changes in pain has been identified, for example circadian variation in the plasma or brain levels of encephalins or beta-endorphin. Inadequate pain management associated with a drug overdose or underdose can be explained in part by the differences in chronobiology of diseases.

Many diseases have a certain time period during which pain is worse than at other times. Arthritis, for example, is a common inflammatory disease of the joints that also runs on a biological clock. Two major varieties of the disease exist, rheumatoid arthritis and osteoarthritis. Rheumatoid arthritis is caused by a disordered immune system that attacks components of the joint. Non-rheumatic arthritis includes a wide range of degenerative diseases. Some of them are associated with the formation of crystal deposits in the joints; others are set off by wear and tear, trauma, or infection. Various forms of rheumatoid arthritis affect millions of people, whereas non-rheumatic arthritis affects most people who live past the age of forty to some extent. These arthritic diseases exhibit profound circadian rhythms in the manifestation and intensity of symptoms. The pain, swelling and stiffness of the joint characteristic of rheumatoid arthritis is more severe in the morning. Osteoarthritis is the most common degenerative joint disease with the weight-bearing joints of the hip, knee, back, toes, and fingers most affected. The temporal pattern of pain and stiffness in osteoarthritis varies from one individual to the next, but it is typically less intense in the morning than in the afternoon or evening.

Ankylosing spondylitis is characterized by swelling and discomfort in the joints of the back. It is an inherited disorder, more common in men than in women. Studies have shown the temporal pattern of ankylosing spondylitis to be similar to that of rheumatoid arthritis, with pain intensity greatest between 6 a.m. and 9 a.m. and least bothersome between noon and 3 p.m. In addition, there is a marked seasonal variation, with symptoms much more frequent in winter than summer.

Patient-controlled analgesia (PCA) pumps have been used by postoperative patients for years, but the complexity of the devices and the substantial risk of overdose usually demands that a nurse supervises the patient during the period of drug delivery. This added expense leads to higher healthcare costs and often makes it more expensive to have a PCA in a home setting. The systems consist, for example, of a pump and a thin, flexible tube or catheter, both of which are surgically placed under the skin. The pump is, for example, implanted in the abdominal area, just above or below the belt line. The catheter connects to the pump and is tunneled under the skin to the site where medication is to be delivered. The pump releases the medication at a set rate, and the medication flows from the pump through the catheter to the site of delivery in the intrathecal space.

WO 00/47253 A1, for example, proposes a method of delivering a medication, wherein a control device enables a patient to select a predetermined quantity of medication.

It is a drawback of the method that the pain has to be noticed by the patient before pain relief medication is provided in response to the pain.

### SUMMARY OF THE INVENTION

It is accordingly an object of the present invention to provide a process to prevent commonly occurring episodes of pain.

The above objective is achieved by a self-learning process for adjusting a dosage of a substance administered to a person by a drug delivery device, the process comprising the following steps:
administration of the substance in a dosage according to at least one recurring sequence,
adjusting the at least one recurring sequence in response to a feedback.

It is an advantage of the present invention that the dosage of the substance according to the recurring sequence responds to variations of the person, for example in experiencing pain, instead of constantly releasing the drug.

The recurring sequence, in the sense of this invention, is meant to be any rhythmic development, including circadian, ultradian, and infradian sequences. Most common will be 24-hour rhythms but, for example, 8-hour rhythms are also of considerable importance in pain therapy. Furthermore, weekly rhythms corresponding to working days and weekends, monthly rhythms corresponding to menstruation cycles, and seasonal rhythms are recurring sequences to which the present invention applies. Preferably, the substance is administered in accordance with a number of recurring sequences, comprising one or more of circadian, ultradian, and infradian sequences, which are superposed. It is an advantage that the amount of medication can be changed, for example slowly, as a function of the season, as may be needed in ankylosing spondylitis pain management.

It is an advantage of the self-learning process according to the invention that the administration of the substance can be adapted to the individual person through adaptation of the recurring sequence, in accordance with the feedback, which feedback is given as an input by the person.

Preferably, the recurring sequence is adjusted with regard to the dosage of the substance in the subsequent cycles of the sequence, in accordance with the feedback. Advantageously, the process according to the invention learns from the person's feedback and is thus able to prevent the person from feeling the need to give the feedback in the subsequent cycles of the recurring sequence. For example, a patient under pain therapy inputs his demand for a stronger analgesic medication owing to his actual sensation of pain as a feedback. According to the inventive self-learning process, the recurring sequence will be adapted so as to provide a stronger analgesic medication in the subsequent cycles of the recurring sequence, thus preventing the occurrence of the pain episode for the patient.

An additional advantage is that to the better timing will cause the person to require less medication, thus decreasing a refill frequency, lowering the cost of medication, and possibly lowering toxicity effects and drug resistance.

It is preferred that a number of preceding cycles of the recurring sequence is considered in determining the adjustment of the recurring sequence. For example, the inventive process learns from the prevailing pain episodes associated with the disease causing the pain. The process is advantageously applicable in chronic disease therapy.

It is furthermore preferred that the recurring sequence is adjusted in consideration of a time delay between the administration of the substance and an occurrence of an effect of the substance on a blood drug level of the person. The consideration of the time delay allows a more precise prevention of the occurrence of, for example, a pain episode in the subsequent cycles of the recurring sequence. Even more preferably, the time delay is adapted in dependence on a course of the recurring sequence, i.e. the time delay itself is adapted in accordance with a time function. For example, the time delay during the night may differ from the time delay during the day. However, the optimum time delay is most preferably adapted on the basis of the person's feedback , so that the time delay is also adapted to the individual person.

In a preferred embodiment, the feedback is a demand on the part of the person to increase, decrease, or maintain the dosage of the substance. The person may, for example, input the demand using a kind of remote control with buttons. The feedback preferably refers to a level of pain relief and/or to an experience of side effects by the person. The feedback may comprise a quantitative estimate of the demand as well. The person can give various inputs, e.g. by pressing buttons: indicating that drug administration should increase little or much to relief pain even more, that drug administration should decrease to reduce side effects as dizziness, sleepiness, numbness etc., or that the dosage is just right. Each step may correspond to a certain medication level up to a certain maximum.

In a further preferred embodiment, the feedback is an evaluation of the effect of the dosage of the substance. For example, the person indicates his actual level of pain experience, for example using a visual analog score (VAS) ranging from 0 to 10. The indicated level of pain experience is compared with a predetermined desired level. This embodiment advantageously provides a more detailed information to the self-learning process on the additional amount of drugs needed and its effect. The VAS scale is used to administer drugs, so the resulting feedback given via the VAS is also caused by the response of the person to the particular drug.

In a further preferred embodiment, an initial sequence for the dosage is preset in accordance with a kind of disease to be treated and/or the kind of substance to be administered, for example considering the drug's half-life time. The recurring sequence of the process takes, for example, an average pain symptom curve as a starting condition. Advantageously, pain relief medication is quickly adjusted to the demands of the person this way. More preferably, the initial sequence is further adapted to the person's characteristics that are commonly used in estimating the desired drug amounts, such as body weight or body area, for example. Further parameters, such as the volume of distribution of the substance, can be readily incorporated in the desired initial sequence. Most preferably, the initial sequence is further adapted to take account of specific information on the person and/or the person's disease in order to provide an advantageously individualized starting condition.

It is also preferred to adjust the dosage of the substance on short notice upon the feedback in order to influence a blood drug level of the person as quickly as possible in the current cycle of the recurring sequence. It is furthermore preferred to reduce the dosage of the substance in order to reduce a blood drug level gradually upon an absence of the feedback by the person.

Preferably, an effect of the administered dosage on a blood drug level of the substance is calculated, and more preferably, a peak blood drug level over the recurring sequence is aligned with a maximum demand of the person in accordance with the person's feedback over the recurring sequence. The amount of drug administered is limited by a threshold value to prevent overdosage, and the described embodiment advantageously ensures that the peak blood drug level will indeed correspond to an actual episode of most severe pain. For example, a patient may have two episodes of pain within a few hours, and the first episode being less painful than the second, it is prevented that during the second episode insufficient pain relief medication is administered to reduce pain experience to acceptable levels. A threshold dosage of the substance preferably complies with a maximum blood drug level of the substance, above which the amount of drug may has a direct effect on the health status of the person.

Another object of the present invention is a drug delivery device comprising:
a dispenser for administering a substance,
a controller for controlling a dosage of the substance in accordance with a recurring sequence,
   wherein the recurring sequence is adjustable by a self-learning process comprising the administration of the substance in a dosage according to at least one recurring sequence and the adjustment of the at least one recurring sequence upon a feedback as described above.

The dispenser is, for example, an external, implantable or transdermal pump containing medication, in particular pain relief medication such as, for example, morphine, opioids, fentanyl, lidocain, etc. The controller communicates with the pump in order to adjust the medication dose. It is an advantage that the medication can be adapted to each individual person through an adjustment of the recurring sequence in a self-learning process. Preferably, the adjustment of the recurring sequence is done in dependence on a number of preceding cycles of the recurring sequence, and/or the administration of the substance is carried out in a number of recurring sequences, comprising one or more of circadian, ultradian, and infradian sequences.

Another object of the present invention is a drug delivery system utilizing a drug delivery device as described above, the system further comprising an input unit that is adapted to receive the feedback of the person and to transfer an input to the drug delivery device. The input unit is preferably a handheld device comprising, for example, buttons or a visual analog scale. The system preferably comprises communication means for establishing communication between the drug delivery device and the input device.

The system preferably comprises a microcomputer for executing calculations with regard to the adjustment of the recurring sequence. Furthermore preferred is that the drug delivery system comprises a storage device in which the feedback and/or the adjustment of the recurring sequence can be stored.

The input transferred from the input unit to the drug delivery device may comprise the feedback, the controller of the drug delivery device being adapted to determine the adjustment of the recurring sequence on the basis of the input. In another embodiment, the input unit is adapted to determine the adjustment of the recurring sequence on the basis of the feedback, and the input transferred to the drug delivery device simply effects the adjustment in the control unit of the drug delivery system. In other words, the necessary calculations for determining the actually necessary adjustments of the recurring sequence may either be executed in the input unit or in the drug delivery device.

These and other characteristics, features and advantages of the present invention will become apparent from the following detailed description, taken in conjunction with the accompanying drawings, which illustrate, by way of example, the principles of the invention. The description is given for the sake of example only, without limiting the scope of the invention. The reference figures quoted below refer to the attached drawings.

### BRIEF DESCRIPTION OF THE DRAWING

FIG. 1 schematically illustrates a drug delivery system according to the present invention.

FIG. 2 schematically illustrates an embodiment of an input device of the drug delivery system according to the present invention.

FIG. 3 is a diagram illustrating a self-learning process according to the present invention.

FIG. 4 illustrates the adjustment of a recurring sequence after a self-learning process according to the present invention.

### DETAILED DESCRIPTION OF EMBODIMENTS

The present invention will be described with respect to particular embodiments and with reference to certain drawings; however, the invention is not limited thereto but only by the claims. The drawings described are merely schematic and non-limiting. In the drawings, the size of some of the elements may be exaggerated and not drawn on scale for illustrative purposes.

Where an indefinite or definite article is used when referring to a singular noun, e.g. “a”, “an”, “the”, this includes a plural of that noun unless something else is specifically stated.

Furthermore, the terms first, second, third and the like in the description and in the claims are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein.

Moreover, the terms top, bottom, over, under, and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

It is to be noted that the term “comprising”, used in the present description and claims, should not be interpreted as being restricted to the means listed thereafter; it does not exclude other elements or steps. Thus, the scope of the expression “a device comprising means A and B” should not be limited to devices consisting only of components A and B. It means that with respect to the present invention, the only relevant components of the device are A and B.

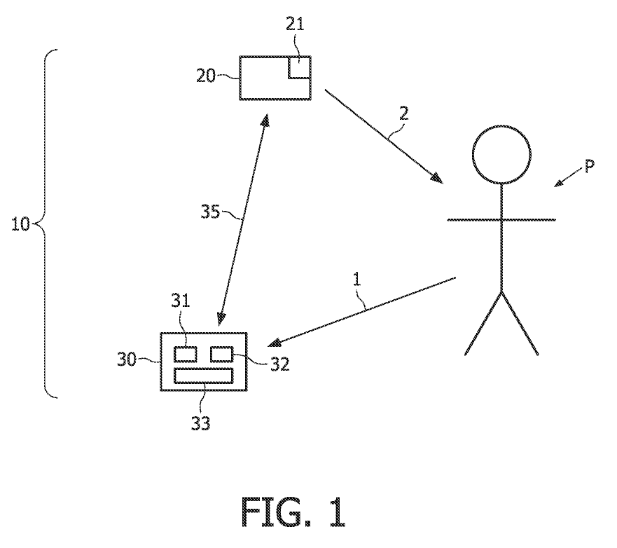

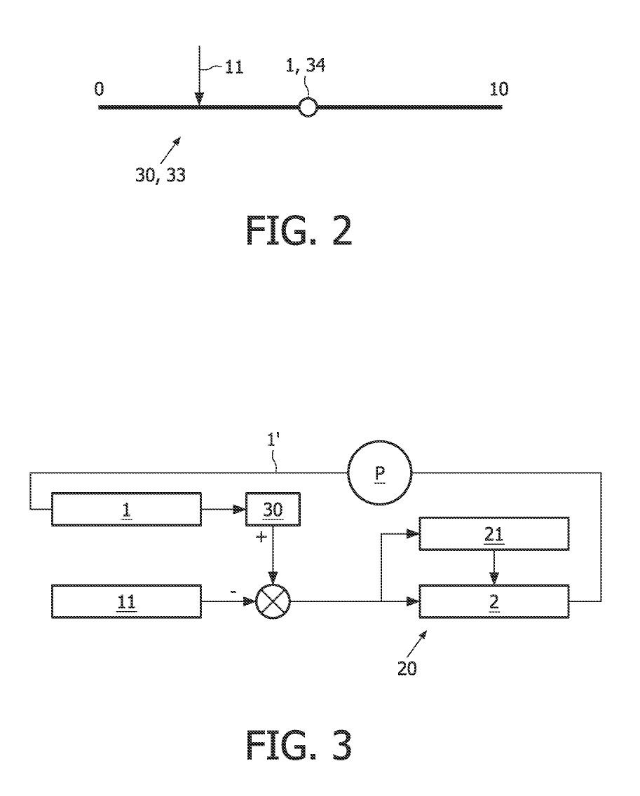

FIG. 1 depicts a drug delivery system 10 comprising a drug delivery device 20 and an input unit 30. The drug delivery device 20 comprises an external, implanted, or transdermal pump of a person P. The drug delivery device is adapted to administer 2 a substance providing a medication, in particular pain relief medication. A dosage of the administration of the substance is controlled in accordance with a recurring sequence 21, which is a kind of drug release profile. The person P gives a feedback 1 via input means 33 to the input unit 30. The input means 33 is, for example, a number of buttons or a visual analog scale as depicted in FIG. 2. The feedback 1 refers to an estimate of the person P as regards an expected effect of the medication.

The input unit 30 further comprises a microcomputer 31 and a storage device 32. An adjustment of the recurring sequence 21 is determined, using the microcomputer 31 for calculation. The feedback 1, preferably comprising both an input value and a time of feedback, is stored on the storage device 32, as is the adjustment of the recurring sequence 21. Those skilled in the art will recognize that the microcomputer and/or the storage device may alternatively be installed on the drug delivery device 20. However, the information relating to an adjusted recurring sequence is transferred to the drug delivery device via communication means, illustrated by arrow 35, which communication means is preferably a wireless connection.

In FIG. 2, an embodiment of the input unit 30 is illustrated by the input means 33, a visual analogue scale which provides more detailed information on the feedback 1 of the person (not depicted) than button inputs do. The level of pain sensation ranges from zero to ten, for example, wherein zero means a complete absence of pain and 10 represents an unbearable pain. A desired setpoint 11 has been set by the person. The setpoint 11 may be reset by the person. The person gives a feedback 1, for example by indicating a position 34 on the visual analog scale 33, said position 34 representing the person's current sensation of pain.

In FIG. 3, a flow diagram illustrates the self-learning process upon the person's P feedback 1, given via the visual analog scale as shown in FIG. 2. The feedback 1 is entered into the input unit 30. The difference between the feedback 1 and the desired setpoint value 11 controls the dosage 2 of the substance administered by the pump of the drug delivery device 20. If the difference is positive, the dosage 2 of the substance will be increased, provided a maximum blood drug level has not yet been reached. Conversely, the dosage 2 is decreased upon a negative difference. Furthermore, the adjustment is applied to the drug release profile of the recurring sequence 21. This drug release profile consists of information on the amount of drug that is desired at each point in time of the recurring sequence 21. So, if the drug release profile perfectly fits the pain experience, the difference is zero and the dosage 2 is based entirely on the stored recurring sequence 21, if not, the recurring sequence 21 will be adjusted. In response to the new, actual experience of the person P, the person P may again provide a feedback 1 when a change is desired. This is represented by the feedback loop 1′. The drug release profile of the next cycle of the recurring sequence 21 is thus an adjustment of the profile of the previous cycle with some correction for differences in pain experience during the previous cycle.

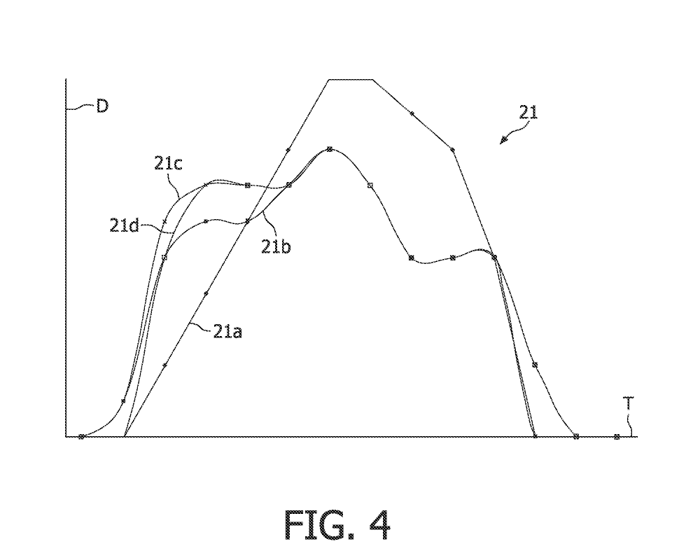

The adjustment of the recurring sequence 21 is illustrated in FIG. 4, which shows a diagram in which the dosage of the substance D is plotted on the vertical axis and the time T is plotted on the horizontal axis. Four subsequent cycles 21a, 21b, 21c and 21d of the recurring sequence 21 are superimposed in order to illustrate the adjustments. The curve of cycle 21a is an initial sequence which is preset based on previous experience in pain relief medication such that an average patient is adequately relieved. The curve of the subsequent cycle 21b is adjusted upon the feedback given by the person during the first cycle of the recurring sequence. Cycle 21b thus shows an enhanced adaptation to the individual pain sensation of the person. The third and fourth cycles 21c and 21d provide an even better adaptation, using the feedback by the person given in the preceding cycles 21a and 21b. The adjustment between the third cycle 21c and the fourth cycle 21d is marginal, which shows that the feedback of the person indicated his/her satisfaction with the medication, i.e. the recurring sequence 21 is well adapted to the individual pain sensation of the person.

The stored data may alternatively be gathered and analyzed by an internal and/or external microcomputer, such that relevant information on rhythms and rhythmic changes in the recurring sequences may be extracted, for example by a pattern recognition technology. The results may then be fed back into the drug delivery system.

1. Self-learning process for automatically adjusting a dosage of a substance administered to a subject by a drug delivery device, the process comprising the following steps:
administering the substance in a dosage in accordance with at least one recurring sequence,
adjusting the at least one recurring sequence in response to a feedback.

2. Process according to claim 1, wherein the recurring sequence is adjusted with regard to the dosage of the substance in the successive cycles of the sequence, in accordance with the feedback.

3. Process according to claim 1, wherein a number of preceding cycles of the recurring sequence is considered for the adjustment of the recurring sequence.

4. Process according to claim 1, wherein the recurring sequence is adjusted in consideration of a time delay between the administration of the substance and an occurrence of an effect of the substance on a blood drug level of the subject.

5. Process according to claim 4, wherein the time delay is adapted in dependence on a course of the recurring sequence.

6. Process according to claim 1, wherein the substance is administered in a number of recurring sequences, comprising one or more of circadian, ultradian, and infradian sequences.

7. (canceled)

8. Process according to claim 1, wherein the feedback is a demand on the part of the subject to increase, decrease, or maintain the dosage of the substance.

9. Process according to claim 1, wherein the feedback is an evaluation of the effect of the dosage of the substance.

10. Process according to claim 1, wherein the feedback relates to a level of pain sensation relief and/or to side effects experienced by the person.

11. Process according to claim 1, wherein an initial sequence for the dosage is preset according to a kind of disease to be treated and/or to the kind of substance to be administered.

12. Process according to claim 11, wherein the initial sequence is further adapted to one or more of: (1) parameters related to the subject, (2) case-specific information on the subject, and (3) the subject's disease.

13. (canceled)

14. Process according to claim 1, wherein the dosage of the substance is reduced so as to reduce a blood drug level upon an absence of the feedback.

15. Process according to claim 1, wherein a peak blood drug level over the recurring sequence is aligned with a maximum demand of the subject in accordance with the feedback over the recurring sequence.

16. (canceled)

17. Drug delivery device, comprising:
a dispenser for administering a substance,
a controller for controlling a dosage of the substance in accordance with a recurring sequence,
wherein the recurring sequence is adjustable by a self-learning process comprising the administration of the substance in a dosage in accordance with at least one recurring sequence and the adjustment of the at least one recurring sequence upon a feedback.

18. Drug delivery device according to claim 17, wherein the adjustment of the recurring sequence is done in dependence on a number of preceding cycles of the recurring sequence.

19. Drug delivery device according to claim 17, wherein the administration of the substance is done in a number of recurring sequences comprising one or more of circadian, ultradian, and infradian sequences.

20. Drug delivery system, comprising a drug delivery device according to claim 17, further comprising an input unit that is adapted to receive the feedback of the subject and to transfer an input to the drug delivery device.

21. Drug delivery system according to claim 20, wherein the input unit is a handheld device.

22. Drug delivery system according to claim 20, further comprising a microcomputer for executing calculations with regard to the adjustment of the recurring sequence.

23. Drug delivery system according to claim 20, further comprising a storage device on which the feedback and/or the adjustment of the recurring sequence can be stored.
